(19) United States
(12) Patent Application Publication
(10) Pub. No.: US 2012/0316143 A1
(43) Pub. Date: Dec. 13, 2012

(54) Ophthalmic composition

(76) Inventor: David B. Soll, Ambler, PA (US)

(21) Appl. No.: 13/507,979

(22) Filed: Aug. 10, 2012

Related U.S. Application Data
(62) Division of application No. 12/589,341, filed on Oct. 22, 2009, now abandoned.

(51) Int. Cl. A61K 31/57; A61P 27/06; A61P 27/02

(52) U.S. Cl. 514/171

(57) ABSTRACT

It has now been found that tetrahydrocortisol alone, when in solution or in the form of nanoparticles, or a combination of compounds where each affects intraocular pressure by a different mechanism of action is especially valuable for the treatment of elevated intraocular pressure and glaucoma, especially in the resistant patient. With such a combination, each compound may be present at a reduced concentration, even to a sub-therapeutic level (i.e., as little as 25% to 50% of the usual therapeutically active dose), thus decreasing or eliminating troublesome side effects while the combined effect on reducing intraocular pressure remains in the therapeutically useful range.

### CROSS REFERENCE

This is a divisional of U.S. Ser. No. 12/589,341, filed Oct. 22, 2009 and claims the benefit of application Ser. No. 61/197,455, filed Oct. 27, 2008.

### BACKGROUND OF THE INVENTION

Glaucoma is an ocular disorder associated with elevated intraocular pressures which are too high for normal function and which may result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by many ophthalmologists to represent the earliest phase of glaucoma.

Many of the drugs formerly used to treat glaucoma, such as pilocarpine and physostigmine, have proven to be not entirely satisfactory. More recently, clinicians have noted that many beta-adrenergic blocking agents are effective in reducing intraocular pressure. The exact mechanism of action in lowering elevated intraocular pressure has not been determined; however it appears to be by a reduction of aqueous production and possibly by some increase in aqueous outflow. Many of these agents, however, also have other activities that are not acceptable for chronic ocular use, e.g. membrane stabilizing activity, which is a local anesthetic effect that prevents the detection of foreign bodies in the eye. These agents are, however, contraindicated in patients with bronchial asthma, atrioventricular heart block, cardiac failure, or hypersensitivity to this medication or any component in the formulation. (S)-1-tert-butylamino((4-morpholino-1,2,5-thiadiazol-3yl)oxy)-2-propanol, which is a beta-adrenergic blocking agent, has been found to reduce intraocular pressure and to be devoid of many unwanted side effects associated with pilocarpine and, in addition, to possess advantages over many other beta-adrenergic blocking agents, e.g. to be devoid of local anesthetic properties, to have a long duration of activity, and to display minimal tolerance. However, such agents, even given topically, may block peripheral beta-adrenergic receptors, which results in a decrease in heart rate and cardiac contractility, as well as respiratory reactions.

Another class of agents used in the treatment of chronic glaucoma are the epinephrines, especially Epifrin® (ophthalmic epinephrine) and dipivefrin, which is a prodrug metabolized to epinephrine in vivo. These drugs work by mainly increasing aqueous outflow. These drugs are contraindicated in angle closure glaucoma and patients who are hypertensive. The safety and efficiency of this class of drugs has not been evaluated in children.

There is another class of agents, referred to as carbonic anhydrase inhibitors, which block or impede the aqueous humor inflow pathway by inhibiting the enzyme carbonic anhydrase. While such carbonic anhydrase inhibitors have been used to treat intraocular pressure by oral, intravenous, or other systemic routes, their activity is not limited to the eye; i.e., they act systemically and thus have the distinct disadvantage of inhibiting carbonic anhydrase throughout the entire body. Such a gross disruption of a basic enzyme system is justified only during an acute attack of alarmingly elevated intraocular pressure or when no other agent is effective.

Topically effective carbonic anhydrase inhibitors have been reported in U.S. Pat. Nos. 4,386,098, 4,416,890, and 4,426,388. The compounds reported therein are 5(and 6)-hydroxy-2-benzothiazolesulfonamides and acyl esters thereof. Benzothiophene-2-sulfonamides, benzenesulfonylthiophene-2-sulfonamides, and thieno(2,3-b)thiopyran-2-sulfonamides are also reported to be carbonic anhydrase inhibitors and said to be topically effective in reducing intraocular pressure in U.S. Pat. Nos. 4,668,697, 4,585,787, and 4,797,413. The carbonic anhydrase inhibitors are not ideal and are handicapped by poor aqueous solubility often requiring formulations of low pH to achieve a pharmacologically useful concentration in solution. Such formulations often cause burning and stinging sensations when applied to the surface of the eye as an eye drop. The low solubility often inhibits the full efficacy of these drugs by limiting their solution concentration.

Another class of compounds reported to be topically effective in lowering intraocular pressure are the prostaglandin F_{2α} (PGF_{2α}) agonists such as latanoprost. Unlike the carbonic anhydrase inhibitors, the prostaglandins are believed to reduce intraocular pressure by increasing the outflow of aqueous humor through an increase in uveoscleral outflow. Compounds of this class, on topical administration, increase pigmentation of the iris and periorbital tissue, resulting in an undesirable side effect: a gradual change in eye color, increasing the amount of brown pigment in the iris by increasing the number of melanosomes in melanocytes. This is most evident in patients with light irises, i.e. blue, green, and hazel.

Another class of compounds of interest are the Rho-kinase inhibitors. Rho-kinase is present in the trabecular meshwork, where inhibition of its action by antagonists affect actin stress fibers and thereby increase aqueous humor outflow. (Inves. Ohpthmol.and Vis. Sci. 42, 1029, 2001.)

Another class of compounds reported to be effective in lowering intraocular pressure are the adenosine A3 receptor antagonists (U.S. Pat. No. 6,528,516, Mini Rev. in Med Chem. 1, No. 4, 339, 2001 and Bioorg. Med. Chem. Letters 14, 3775, 2004.)

A combination of two ocular antihypertensive drugs has been used to increase the efficacy needed for the control of intraocular pressure in the more resistant patient. The combination of a beta-adrenergic antagonists and a carbonic anhydrase inhibitor was particularly useful in the treatment of such patients and was more efficacious than either agent alone. Thus, the combination of the two drugs reduces the intraocular pressure below that obtained by either medicament individually. In the case of this combination, carbonic anhydrase inhibitor and a beta-andrenergic blocking agent, both drugs work by the same mechanism, that is, a decrease in aqueous humor formation.

There exists a need to treat the patient population with a minimal dosage of the ocular antihypertensive agents, thereby minimizing the possibility of the occurrence of undesirable effects which become apparent with chronic treatment or at higher dosage.

### SUMMARY OF THE INVENTION

It has now been found that tetrahydrocortisol is especially valuable for the treatment of elevated intraocular pressure, especially in the resistant patient, when the THC is in solution or in the form of nanoparticles.

It has also now been found that a combination of compounds where each affects intraocular pressure by a different mechanism of action is especially valuable for the treatment of elevated intraocular pressure, especially in the resistant patient. With such a combination, each compound may be present at a reduced concentration, even to a sub-therapeutic level (i.e., as little as 25% to 50% of the usual therapeutically active dose), thus decreasing or eliminating troublesome side effects while the combined effect on reducing intraocular pressure remains in the therapeutically useful range.

### DETAILED DESCRIPTION OF THE INVENTION

The ophthalmic compositions of this invention comprise a sub-therapeutic to a therapeutically effective amount of A (an ocular antihypertensive drug) with (B) (a) tetrahydrocortisol (THC), (b) an inhibitor of cortisol synthesis, (c) a cortisol receptor antagonist, or (d) a potassium channel blocker such as one disclosed in U.S. Pat. No. 7,414,067 (Boyd et al.), or a pharmaceutically acceptable or a pharmaceutically acceptable salt, enantiomer, diastereomer, or mixtures thereof.

The ocular antihypertensive drug (A) of this novel composition can include: 1) carbonic anhydrase inhibitors such as dorzolamide and brinzolamide; 2) beta-adrenergic blocking agents including timolol, betaxolol, levobunolol and metipranolol; 3) prostaglandin F_{2α} agonists, both natural products and synthetic analogs, including latanoprost, unoprostrone isopropyl, travoprost, and bimatoprost; 4) a selective alpha adrenergic agonist such as brimonidine and clonidine, 5) epinephrine, 6) Rho-kinase inhibitors, and 7) adenosine A3 receptor antagonists. Alpha agonists primarily affect aqueous formation. The preferred carbonic anhydrase inhibitor is dorzolamide. The preferred beta-adrenergic antagonist is timolol. The preferred prostaglandin agonist is latanoprost. The preferred alpha adrenergic agonist is brimonidine. The preferred Rho-kinase inhibitor is Y 27632. The preferred adenosine A3 receptor antagonist is a 1,2,4-triazolo-(1,5c)pyrimidine.

Tetrahydrocortisol, especially the 3-alpha-5-beta isomer, has been shown to lower intraocular pressure (IOP) in rabbits made ocularly hypertensive with dexamethasone (Southren Weinstein el al., Invest. Ophthalmol. Vis. Sci. 28, 901 (1987) and to be useful in the prevention of the elevation in intraocular pressure resulting from treatment with glucocorticoids (U.S. Pat. No. 5,358,943, Clark et al.). 3-α-5-β-Tetrahydrocortisol has been shown to lower intraocular pressure in patients when applied topically as a 1% suspension. (J. Ocul. Pharmacol. 10,385 (1994) and in U.S. Pat. No. 4,997,826 (Southren et al.)). THC may affect intraocular pressure by preventing changes in the trabecular meshwork cytoskeleton caused by glucocorticoids, such as cortisol (Invest. Opthalmol. Vis. Sci. 37, 805-13(1996)). In line with this proposal is the observation that decreasing cortisol formation in the eye by inhibition of its synthesis reduces IOP. Carbenoxolone treatment resulted in a 20% decrease in intraocular pressure in normal subjects (Invest. Ophthalmol. 41, 1609-1638 (2000)) and in patients with ocular hypertension (Q.J. Med. 96:481-490 (2003)). However, THC has not been used with other ocular antihypertensive as an approach to enhanced efficacy for resistant glaucoma. Such a combination takes advantage of different mechanisms affecting intraocular pressure. Such a combination, where each component reduces intraocular pressure by an independent mechanism has now been unexpectantly found to lower intraocular pressure at a dose below that required for each single entity, thereby allowing novel formulations which can decrease or eliminate the undesirable side effects caused by the therapeutic dose of each entity when given separately. Tetrahydrocortisol has been used topically only as a suspension. When the formulation of the present combination is in the form of a solution both components, including the THC, are in solution or as nanoparticles and thus fully available for passage through the cornea.

Inhibitors of cortisol synthesis of the invention function via inhibition of 11-β-hydroxysteroid dehydrogenase. They include compounds such as those described in U.S. Pat. Nos. 7,332,524 and 7,304,081 and are exemplified by the adamantyl acetamides such as N-(5-hydroxytricyclo-(3.3.1.13)deca-2-yl)-α,α-dimethylbenzene acetamide and the phenylpyrrolidines and related compounds such as 1-((1-(4-chlorophenyl)cyclopropyl)carbonyl)-2-phenylpyrrolidine or the corresponding 2,3-dihydrospiro(indene-1,4-piperidine). When used in the ophthalmic formulations of the invention, the inhibitors of cortisol synthesis comprise 0.05 to 5% (w/w), preferably 1 to 2% of the formulation and are administered 1 to 4 times a day, preferably 1 to 2 times a day.

The glucocorticoid antagonists are represented by mifepristone. When used in the ophthalmic formulations of the invention, the glucocorticoid antagonists comprise 0.05 to 5 (w/w), preferably 1 to 2% of the formulation and are administered 1 to 4 times a day, preferably 1 to 2 times a day.

Preferred potassium channel blockers are calcium activated potassium channel blockers. More preferred potassium channel blockers are high conductance, calcium activated potassium (Maxi-K) channel blockers.

One embodiment of the invention is an ophthalmically effective amount of a composition comprising tetrahydrocortisol in solution or as nanoparticles as the only ocular antihypertensive compound.

Another embodiment of the invention is nanoparticle size tetrahydrocortisol.

Another embodiment of the invention is a method of treating a patient with elevated intraocular pressure or glaucoma, wherein said patient is a mammal, especially man, which comprises administering to said patient an ophthalmically effective amount of a composition comprising tetrahydrocortisol in solution or in the form of nanoparticles. The ophthalmically effective amount typically comprises 3-8% (w/w), preferably 5%, and is administered 2-4 times a day single drop schedule.

One embodiment of the invention is ophthalmically effective amount of a composition comprising an ocular antihypertensive compound A and B (tetrahydrocortisol, an inhibitor of cortisol synthesis, a cortisol receptor antagonist, or a potassium channel blocker).

Another embodiment of the invention is a method of treating a patient with elevated intraocular pressure or glaucoma, wherein said patient is a mammal, especially man, which comprises administering to said patient an ophthalmically effective amount of a composition comprising an ocular antihypertensive compound A and B (tetrahydrocortisol, an inhibitor of cortisol synthesis, a cortisol receptor antagonist, or a potassium channel blocker). Said administration may be concomitant or sequential.

The combinations disclosed herein are effective either by co-administration of the medicaments as a single composition or as a sequential therapy achieved by prior administration of one medicament followed by administration of the other. The second medicament may be applied after tearing ceases or after 30 seconds. In the case of sequential administration of the individual drugs, either may be given first followed by the second. The use of a single composition containing both medicaments is preferred.

The topical formulations of the invention include: 1) carbon anhydrase inhibitors, especially dorzolamide, and 3-alpha-5-beta-tetrahydrocortisol or a potassium channel blocker; 2) a beta-adrenergic blocking agents, especially timolol, and 3-alpha-5-beta tetrahydrocortisol or a potassium channel blocker; 3) a prostaglandin Fₗₐ agonist, especially latanoprost, and 3-alpha-5-beta-tetrahydrocortisol or a potassium channel blocker; and 4) a selective alpha-adrenergic agonist, especially apraclonidine or brimonidine, and 3-alpha-5-beta-tetrahydrocortisol or a potassium channel blocker, 5) epinephrine and 3-alpha-5-beta-tetrahydrocortisol or a potassium channel blocker 6) a Rho-kinase inhibitor and 3-alpha-5-beta-tetrahydrocortisol or a potassium channel blocker, and 7) an adenosine A3 receptor antagonist and 3-alpha-5-beta-tetrahydrocortisol or a potassium channel blocker.

The combination of dorzolamide and tetrahydrocortisol is formulated at near neutral pH and with low viscosity, thereby eliminating the stinging and burning side effects common with the commercial dorzolamide 2% at pH 5.6. The lower pH is primarily responsible for the uncomfortable side effect of burning. Brimzololamide is a suspension and does not penetrate the corneal barrier as efficiently as Trusopt®.

For the carbonic anhydrase inhibitor/THC formulation, the carbonic anhydrase inhibitors can be selected from dorzolamide and brinzolamide, or an ophthalmolgically acceptable salt thereof. The ophthalmic formulations of the invention comprise about 0.05 to 5% (w/w) carbonic anhydrase inhibitor, usually about 0.5 to 3% (w/w), and about 0.05 to 5% (w/w) THC usually about 0.5 to 3% (w/w) to be administered on a 1 to 2 times a day single drop schedule.

For the beta-adrenergic antagonist/THC formulation, the beta-adrenergic antagonist is selected from betaaxolol, carteolol, levobunolol, metipranolol, and timolol, or an ophthalmogically acceptable salt thereof. The ophthalmic formulations of the invention comprise about 0.01 to 1% (w/w) of the beta-adrenergic antagonist, preferably about 0.1 to 0.5% (w/w), in combination with about 0.05 to 5% (w/w) of THC, usually about 0.5 to 3% (w/w), and is administered on a 1 to 2 times a day single drop schedule.

For the prostaglandin agonist/THC formulation, the prostaglandin is selected from bimatoprost, latanoprost, travoprost, and unoprostone, or any ophthalmologically acceptable salt thereof. The novel ophthalmic formulations of the invention comprise about 0.0001 to 1% (w/w) of the prostaglandin, preferably 0.001 to 0.5% (w/w), in combination with about 0.05 to 5% (w/w) of THC, usually about 0.5 to 3% (w/w), and is administered on a 1 to 2 times a day single drop schedule.

For the selective alpha adrenergic agonist/THC formulation, the selective alpha adrenergic agonist is selected from brimonidine and clonidine, or any ophthalmologically acceptable salt thereof. The novel ophthalmic formulations of the invention comprise about 0.015 to 5% (w/w) of the selective alpha adrenergic agonist, preferably 0.5 to 2% (w/w), in combination with about 0.05 to 5% (w/w) of THC, usually about 0.5 to 3% (w/w), and is administered on a 2 to 4 times a day single drop schedule.

For the epinephrine/THC formulation, the epinephrine is selected from ophthalmic epinephrine and dipivefrin. The novel ophthalmic formulations of the invention comprise about 0.05 to 10% (w/w) of the epinephrine, preferably 0.1 to 5% (w/w), in combination with about 0.05 to 5% (w/w) of THC, usually about 0.5 to 3% (w/w), and is administered on a 2 to 4 times a day single drop schedule.

For the Rho-kinase inhibitor/THC formulation, the Rho-kinase inhibitor is preferably Y 27632. The novel ophthalmic formulations of the invention comprise about 0.2 to 3% (w/w) of the Rho-kinase inhibitor, preferably 0.5 to 5% (w/w), in combination with about 0.05 to 5% (w/w) of THC, usually about 0.5 to 3% (w/w), and is administered on a 2 to 4 times a day single drop schedule.

For the adenosine A3 receptor antagonist/THC formulation, the adenosine A3 receptor antagonist is preferably a 1,2,4 triazolo (1,5-c)pyrimidine. The novel ophthalmic formulations of the invention comprise about 0.2 to 5% (w/w) of the adenosine A3 receptor antagonist, preferably 1 to 3% (w/w), in combination with about 0.05 to 5% (w/w) of THC, usually about 0.5 to 3% (w/w), and is administered on a 2 to 4 times a day single drop schedule.

Formulations analogous to those above may be prepared, but replacing THC with an inhibitor of cortisol synthesis, a cortisol receptor antagonist, a potassium channel blocker. Ophthalmic formulations of this compound may contain from 0.01 to 5% and especially 0.5 to 2% of the potassium channel blocker. For a single dose, from between 0.001 to 5.0%, preferably 0.005 to 2.0%, and especially 0.005 to 1.0% of the potassium channel blocker may be applied to the eye.

For topical ocular administration, the formulations of the invention may take the form of solutions, gels, ointments, suspensions, or solid inserts, formulated so that a unit dosage comprises a therapeutically effective amount of each active component, even if the amount is less than if the components were given individually.

Typical ophthalmologically acceptable carriers for the novel formulations are, for example, water, mixtures of water and water-miscible solvents such as lower alkanols or aralkanols, vegetable oils, polyalkylene glycols, petroleum based jelly, ethyl cellulose, ethyl oleate, carboxymethylcellulose, polyvinylpyrrolidone, isopropyl myristate and other conventionally employed pharmaceutically acceptable carriers, including the various cyclodextrins. The pharmaceutical preparation may also contain non-toxic auxiliary substances such as emulsifying, preserving, or wetting agents, bodying agents and the like, for example, polyethylene glycols 200, 300, 400 and 600, carbowaxes 1000, 1,500, 4,000, 6,000, and 10,000, antibacterial components such as quaternary ammonium compounds, phenylmercuric salts known to have cold sterilizing properties and which are non-injurious in use, thimerosal, benzalkonium chloride, methyl and propyl paraben, benzyldodecinium bromide, benzyl alcohol, phenylethanol, buffering ingredients such as sodium chloride, sodium borate, sodium acetate, or gluconate buffers, and other conventional ingredients such as sorbitan monolaurate, triethanolamine, polyoxyethylene sorbitan monopalmitylate, dioctyl sodium sulfosuccinate, monothioglycerol, thiosorbitol, ethylenediamine tetra-acetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles, and the like.

The formulation may also include a gum such as gellan gum at a concentration of 0.1 to 2% by weight so that the aqueous eye drops gel on contact with the eye, thus providing the advantages of a solid ophthalmic insert, as described in U.S. Pat. No. 4,861,760.

The pharmaceutical preparation may also be in the form of a solid insert such as one which after dispensing the drug remains essentially intact, as described in U.S. Pat. Nos. 4,256,108; 4,160,452; and 4,265,874; or a bio-erodible insert that is either soluble in lachrymal fluids, or otherwise disintegrates, as described in U.S. Pat. No. 4,287,175 or EP 0,077,261.

The formulation may also be composed of low micron or nano sized particles of THC thereby providing a significant advantage in terms of drug uptake into the eye.

Nanoparticles are compounds with an average diameter of between 1 and 100 nanometers. They may be prepared by methods well known in the art; for example by milling a dry powder of the compound or a slurry thereof.

Several cyclodextrins may be used, including randomly methylated-beta-cyclodextrin, 2-hydroxypropyl-beta-cyclodextrin, and sulfobutylether-beta-cyclodextrin. For each formulation, a phase solubility study is performed to determine the exact amount of cyclodextrin to solubilize the THC and dorzolamide or another intraocular pressure lowering medication or its ophthalmologically acceptable salt at the near neutral pH of 6 to 7.5.

The compounds of the invention may be in the form of a pharmaceutically acceptable salt thereof. Other salts may, however, be useful in the preparation of the compounds according to the invention or of their pharmaceutically acceptable salts. When the compound of the present invention is acidic, suitable “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary and tertiary amines, substituted amines including naturally occurring substituted amines, cyclic amines, and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the invention is acidic, suitable derivatives such as esters and amides are also included.

When a compound of the invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic, and the like.

All cited patents and publications are incorporated herein by reference in their entirety.

The invention can be further exemplified by the example, which is intended to be illustrative and not limiting,

### EXAMPLE 1

An excess of tetrahydrocortisol is added to aqueous phosphate buffer pH 8 (2.05 M). The buffer contains 0% to 20% (w/v) cyclodextrin, benzalkonium chloride (2.02% w/v), ethylenediaminetetraacetic acid (EDTA) (0.1% w/v), and hydroxylpropylmethylcellulose (0.1%). The pH is adjusted to 7.5 with lON NaOH. The mixture is autoclaved for 20 minutes at 120° C. and allowed to cool and stand at room temperature (27° C.) for 7 days. The suspension is then filtered and 10% excess of cyclodextrin added. This stock solution of tetrahydrocortisol is then used to prepare the eye drop formulations used for the combination formulations of the invention.

A dorzolomide solution, similarly prepared, is mixed with the stock solution of THC to yield final formulations containing 1% dorzolamide/1% THC, 1% dorzolamide/2% THC, 1.5% dorzoamide/1% THC, 1.5% dorzolamide/2% THC, and 2% dorzolomide/2% THC. The tested viscosity for the cyclodextrin/dorzolamide formulations ranges from 3 to 5 cPs, and each is adjusted to 10 cPs (mPa·s) as measured at room temperature on a Brookfield viscometer, the viscosities of water being 1 cPs and of Trusopt® about 100 cPs.

What is claimed is:

1. A composition comprising an ocular antihypertensive compound A,
wherein A is selected from the group consisting of 1) carbonic anhydrase inhibitors; 2) beta-adrenergic blocking agents including timolol, betaxolol, levobunolol, and metipranolol; 3) prostaglandin F_{2α} agonists, both natural products and synthetic analogs, including latanoprost, unoprostrone isopropyl, travoprost, and bimatoprost; 4) a selective alpha adrenergic agonist such as brimonidine and clonidine, 5) epinephrine, 6) Rho-kinase inhibitors, and 7) adenosine A3 receptor antagonist;
compound B, which is an inhibitor of cortisol synthesis, a corisol receptor antagonist, or a potassium channel blocker);
and an ophthalmologically acceptable carrier therefor.

2. A composition of claim 1 where the carbonic anhydrase inhibitor is (S,S)-5,6-dihydro-4(ethylamino)-6-methyl-4-H-thieno-(2,3-b)-thiopyran-2-sulfonamide-7,7-dioxide, dorzolamide, or brinzolamide or an ophthalmologically acceptable salt thereof.

3. A composition of claim 1 where the beta-adrenergic blocking agent is timolol, betaxolol, levobunolol, or metipranolol.

4. A composition of claim 1 where the prostaglandin F_{2α} agonist is latanoprost, unoprostrone isopropyl, travoprost, or bimatoprost.

5. A composition of claim 1 where the selective alpha adrenergic agonist is as brimonidine or clonidine.

6. A composition of claim 1 where the epinephrine is ophthalmic epinephrine or dipivefrin.

7. A composition of claim 1 where the Rho-kinase inhibitor is Y 27632 and the adenosine A3 receptor antagonist is a 1,2,4 triazolo (1,5-c)pyrimidine.

8. A composition of claim 1 comprising 0.05 to 5% of an adamantyl acetamide or a phenylpyrrolidine and 0.05 to 5% of a carbonic anhydrase inhibitor or its ophthalmologically acceptable salt.

9. A composition of claim 1 comprising 0.05 to 5% of an adamantyl acetamide or a phenylpyrrolidine and 0.01 to 1% of a beta andenergic antagonist or an ophthalmologically acceptable salt thereof.

10. A composition of claim 1 wherein the beta-adrenergic antagonist is (S)-(-)-1)-(tert-butylamino)-3-((4morpholino-1,2,5-thiadiazol-3-yl)oxypropanol or an ophthalmologically acceptable salt thereof.

11. A composition of claim 1 comprising 0.05 to 5% of of an adamantyl acetamide or a phenylpyrrolidine and 0.001 to 0.5% of a prostaglandin F_{2α} agonist or an ophthalmologically acceptable salt thereof.

12. A composition of claim 10 where the prostaglandin F_{2α} agonist is 13,14-dihydro-17-phenyl-18,19,20-trinor-PGF_{2α} isopropylester.

13. A composition of claim 5 where the alpha-adrenergic agonist is 5-bromo-N-(4.5-dihydro-1H-imidazol-2-yl)-6-quinoxalinamine or its ophthalmologically acceptable salt.

14. A composition of claim 1 where the ophthalmologically acceptable carrier comprises a cyclodextrin.

15. A composition of claim 14 where the cyclodextrin is selected from randomly methylated-beta-cyclodexrin, 2-hydroxypropyl-beta-cyclodextrin, sulfobutylether-beta-cyclodextrin, and tetradecasulfate-beta-cyclodextrin.

16. A composition of claim 15 wherein the cyclodextrin is randomly methylated-beta-cyclodextrin.

17. An ophthalmically effective amount of a composition comprising of an adamantyl acetamide or a phenylpyrrolidine which comprises 1-2% (w/w) N-(5-hydroxytricyclo-(3.3.1.13)deca-2-yl)-α,α-dimethylbenzene acetamide, 1-((1-(4-chlorophenyl)cyclopropyl)carbonyl)-2-phenylpyrrolidine , or 2,3-dihydrospiro(indene-1,4-piperidine), which is administered 1-4 times a day single drop schedule.

18. A method of treating a patient with elevated intraocular pressure or glaucoma, wherein said patient is a mammal, especially man, which comprises administering to said patient an ophthalmically effective amount of a composition of claim 1 administered 2-4 times a day single drop schedule.
